(19) United States
(12) Patent Application Publication
(10) Pub. No.: US 2008/0176326 A1
(43) Pub. Date: Jul. 24, 2008

(54) Delivery of high cell mass in a syringe and related methods of cryopreserving cells

(75) Inventor: Samih Yaghmour, Thousand Oaks, CA (US)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: Amgen Inc.

(21) Appl. No.: 12/003,964

(22) Filed: Jan. 3, 2008

Related U.S. Application Data
(63) Continuation of application No. 11/186,853, filed on Jul. 22, 2005, now abandoned.
(60) Provisional application No. 60/590,437, filed on Jul. 23, 2004.

(51) Int. Cl. C12N 5/00; C12M 1/36

(52) U.S. Cl. 435/374; 435/286.4; 435/375; 435/379; 435/383

(57) ABSTRACT

This invention relates to methods and apparatus for cryopreserving biological materials for extended periods of time. In an exemplary embodiment, the method comprises suspending biological materials in a cryosolution, freezing the biological materials in the apparatus, and removing the frozen biological materials from the apparatus to thaw them for use. In another embodiment, a cell cryopreservation solution is provided which includes 20% Dimethyl Sulfoxide (DMSO) to maintain the viability of cells upon freezing, storage, and thawing. The media can be used for cryopreservation of a wide variety of different cell types from various sources. In addition, an apparatus that facilitates the storage of cells and the subsequent removal of the cells for thawing to permit substantially direct inoculation of a bioreactor is disclosed. Cells frozen using a method according to the present disclosure have been shown to have approximately a 90% survival rate, which is significantly higher than other cryopreservation methods.

### CROSS-REFERENCE TO RELATED APPLICATION

This patent application claims the benefits of priority under 35 U.S.C. § 119 to U.S. Provisional Patent Application No. 60/590,437, entitled DELIVERY OF HIGH CELL MASS IN A SYRINGE AND RELATED METHODS OF CRYOPRESERVING CELLS, filed on Jul. 23, 2004, the entirety of which is incorporated herein by reference.

### DESCRIPTION OF THE INVENTION

1. Field of the Invention

Embodiments of this invention relate generally to a method of using a syringe to deliver a high cell mass of cryopreserved cells to a bioreactor without the need for cell expansion, and to related methods of preserving biologically active materials in the field of biotechnology. More particularly, embodiments of the processes described herein relate to, for example, cryopreserving biological materials for extended periods of time, and may facilitate substantially direct inoculation of a bioreactor with the cryopreserved materials.

2. Background of the Invention

The field of biotechnology involves the manipulation and/or genetic engineering of living organisms, such as mammalian cells, to produce new cell lines that aid in the production of biologically active products. These products may include, but are not limited to, hormones, growth factors, interleukins, cytokines, and immunoglobulins. The development of new cell lines, through manipulation and/or genetic engineering, generally involves large investments of time and resources. Thus, the successful preservation of newly developed cells and cell lines is important to research and to the development of many biological products. Furthermore, the process of preserving the cells must not, in itself, damage or destroy the cells.

The establishment of cell banks that store the newly developed cell lines is therefore critical to the field of biotechnology. The cell bank system, as a means of preserving newly developed cell lines, assures that the cell line is preserved, its integrity is maintained, and a sufficient supply of the cell line is readily available for use. Furthermore, cell banking may be preferred because it protects the preserved cell lines from, among other things, genotypic drift due to genetic instability, senescence, transformation, phenotypic instability due to selection and differentiation, viral or microbial contamination, and cross-contamination by other cell lines.

Conventional methods of preserving cells involve a technique known as cryopreservation. Cryopreservation can broadly be defined as lowering the temperature of living structures and biochemical molecules to the point of freezing and beyond, where no physical or chemical changes will occur, for the purposes of storage and future recovery of the material in its pre-frozen, viable condition. In current practice, cells are harvested, suspended in a storage solution, and then frozen for preservation. When the cells are needed, they are then thawed and re-cultured in growth media at 37° C. The challenge to cells during cryopreservation is not their ability to endure storage at low temperatures; rather, it is the lethality of an intermediate zone of temperature (e.g., −15 to −60° C.) that cells must traverse twice, once during cooling and once during warming. See Peter Mazur, Freezing of living cells: mechanisms and implications, 247 AMERICAN JOURNAL OF PHYSIOLOGY 125, 142 (1984). As cells are cooled to approximately −5° C., both the cells and surrounding medium remain unfrozen and supercooled. As the cells are further cooled, between approximately −5° C. and approximately −15° C., ice begins to form in the external medium. However, the cells' contents remain unfrozen and supercooled. The supercooled water in the cells has, by definition, a greater chemical potential than that of water in the partially frozen extracellular solution. Thus, water flows out of the cells osmotically and freezes outside the cells. The subsequent physical events in the cells depend on the cooling rate. Rapid cooling minimizes the solute concentration effects as ice forms uniformly, but leads to more intracellular ice. In contrast, slow cooling results in a greater loss of water from the cell and less internal ice, but increases the solution effects. An optimum homogenous cooling rate of 1° C. per minute is usually preferred.

At least some current methods used to cryopreserve cells include the practice of adding animal serum (e.g., fetal calf serum (FCS)) as well as cryopreservative agents (CPAs) to the freeze media/cell storage solution. Traditionally, animal serum has been used for the preservation of cells as it stabilizes cell membranes, and protects the intracellular content from high solute effects. However, due to concerns surrounding animal diseases such as Bovine Spongiform Encephalopathy (i.e., Mad Cow Disease), the addition of animal serum may, in certain instances, expose preserved cells to a source of undesirable contamination.

The clinical and commercial application of cryopreservation for cells may be limited by the ability to recover a significant number of viable cells. For example, current methods of cryopreserving cells yield an insufficient number of cells to directly inoculate a 20 liter bioreactor. Since the number of viable cells recovered from thawing the cryopreserved cells is insufficient, the cells must be subjected to cell culture expansion to produce additional cells until there are enough cells to inoculate the 20 liter bioreactor. The current process of cell culture expansion prior to inoculating such a reactor takes approximately two to four weeks, depending on the cell line. As the expansion process is considered time consuming, labor intensive, and a source of contamination, banking and preservation of high cell mass is becoming increasingly important in the field of biotechnology.

Current methods of preserving large numbers of cells include the use of cryobags to store the cells during freezing. Cryobags have been used to store larger volumes of cells at conventional densities. However, cryobags possess many drawbacks that limit their versatility when used for cryopreservation of cells. For example, the cryobags are subject to potentially experiencing temperature gradients across the sample that leads to non-homogeneous cooling rates. A homogeneous cooling rate is vital to the success of the preservation process. Additionally, cryobags must be frozen in special controlled-rate freezers to prevent material heat shock and bag rupture during cooling. They may also become brittle once the temperature is lowered below the glass transition point of the bag's material, leading to break or rupture during handling and storage. Cryobags are usually thawed in water baths, which can lead to unwanted cell damage and/or contamination.

Thus, there is a need for a cryopreservation process that stabilizes cells during freezing, protects cells from damage, is non-toxic, allows for freezing cells at a high density, allows for rapid recovery of the frozen cells, reduces the potential of external contamination, and is suitable for a wide range of cell types in a wide variety of cell culture and clinical applications.

### SUMMARY OF THE INVENTION

Embodiments of the invention provide apparatus and procedures for freezing and thawing a large volume of cells, e.g., cell masses of between approximately 3.0×10⁸ cells and approximately 5.0×10⁹ cells, that are suitable for rapid expansion upon thawing. The present invention also permits cryopreservation of the large volume of cells at higher densities (e.g., between approximately 3.0×10⁷ cells/ml and approximately 5.0×10⁸ cells/ml) both with and without an animal serum. Freezing at such densities is accomplished through the addition of permeating cryoprotectants to the freeze media in greater than normal or high concentrations. In addition, the present invention permits substantially direct inoculation of a bioreactor with the frozen cells.

In accordance with an aspect of the present invention, an apparatus for storing and dispensing cryopreserved cells includes a body having an open first end and an open second end, a first cap configured to removably attach to the open first end, a second cap configured to removably attach to the open second end, a plunger portion contained within the body and adjacent to one of said open ends, and a plunger rod configured to be connected to the plunger portion, wherein at least a portion of the apparatus is made from a biocompatible material.

Another aspect of the present invention includes a method of rapidly freezing cells. The method includes acquiring a desired quantity of cells for cryostorage, suspending the acquired cells in chilled freeze media containing a permeating cryoprotectant, wherein the freeze media is at a temperature of approximately 0° C. to 4° C., placing the cells and freeze media in an apparatus configured to store and dispense cryopreserved cells, wherein at least a portion of the apparatus is made from a biocompatible material, and rapidly cooling the apparatus containing the cells and chilled freeze media to a temperature of −130° C. or below at a rate of approximately 8° C./minute.

Yet another aspect of the present invention includes a method of rapidly thawing cryopreserved cells. The method includes retrieving a storage apparatus containing frozen media and cells having an approximate temperature of −130° C. or below, and transferring the frozen media and cells from the storage apparatus to a thawing receptacle containing growth media at a temperature of approximately 37° C. to thaw the cells.

A further aspect of the present invention includes a method of cryostoring cells. The method includes acquiring a desired quantity of cells for cryostorage, placing the acquired cells in chilled freeze media containing a permeating cryoprotectant, storing the cells and freeze media in an apparatus suitable for cryostorage, wherein the apparatus is configured to store and dispense cryopreserved cells and includes a body having an open first end and an open second end, a first cap configured to removably attach to the open first end, a second cap configured to removably attach to the open second end, a plunger portion contained within the body and adjacent to one of the open ends, and a plunger rod configured to be connected to the plunger portion, wherein at least a portion of the apparatus is made from a biocompatible material. The method also includes the step of cooling the apparatus to an approximate temperature of −130° C. or below.

Another aspect of the present invention includes a method for inoculating a bioreactor with cryopreserved cells. The method includes acquiring a desired quantity of cells for cryostorage, placing the acquired cells in chilled freeze media containing a permeating cryoprotectant, storing the cells and freeze media in an apparatus configured to store and dispense cryopreserved cells, wherein the apparatus includes a body having an open first end and an open second end, a first cap configured to removably attach to the open first end, a second cap configured to removably attach to the open second end, a plunger portion contained within the body and adjacent to one of the open ends, and a plunger rod configured to be connected to the plunger portion, wherein at least a portion of the apparatus is made from a biocompatible material. The method further includes cooling the apparatus to an approximate temperature of −130° C. or below, subsequent to cooling the apparatus to an approximate temperature of −130° C. or below, transferring the frozen media and cells' from the apparatus to a thawing vessel containing growth media at a temperature substantially warmer than 0° C., and inoculating a bioreactor with the cells from the thawing vessel.

Yet another aspect of the present invention includes a composition for cryopreserving a large cell mass at a high density. The composition includes a freeze media including a permeating cryoprotectant, wherein the concentration of the permeating cryoprotectant is sufficient to permit the cells to be stored at a density greater than 1.5×10⁸ cells/ml; and a large volume of cells, between approximately 3.0×10⁸ cells and approximately 5.0×10⁹ cells, to be stored.

Another aspect of the present invention includes a method of freezing a large cell mass at a high density. The method includes suspending a large cell mass in a freeze media containing a permeating cryoprotectant, wherein the concentration of the permeating cryoprotectant is sufficient to permit the cells to be stored at a density greater than 1.5×10⁸ cells/ml, placing the cells and freeze media in a storage apparatus, and cooling the cells and freeze media to a temperature at or below approximately −130° C.

A further aspect of the present invention includes a method of rapidly thawing a large, frozen cell mass. The method includes retrieving a storage apparatus containing a frozen cell mass and freeze media, and transferring the frozen cell mass and freeze media from the storage apparatus to a thawing vessel containing growth media at a temperature of approximately 37° C. to thaw the cells.

Another aspect of the present invention includes a composition for cryopreserving a large cell mass at a high density. The composition includes a freeze media including 20% Dimethyl Sulfoxide (DMSO), wherein the freeze media does not include animal serum, and wherein the concentration of the DMSO is sufficient to permit the cells to be stored at a density greater than 3.0×10⁷ cells/ml, and a large volume of cells to be stored.

Yet another aspect of the present invention includes a method of freezing a large cell mass at a high density. The method includes suspending a large cell mass in a freeze media containing 20% DMSO, wherein the freeze media does not include animal serum and wherein the concentration of the DMSO is sufficient to permit the cells to be stored at a density greater than 3.0×10⁷ cells/ml, placing the cells and freeze media in a storage apparatus, and cooling the cells and freeze media to a temperature at or below approximately −130° C.

Additional objects and advantages of the invention will be set forth, in part, in the description which follows and, in part, will be obvious from the description, or may be learned by practice of the invention. The objects and advantages of the invention will be realized and attained by means of the elements and combinations, particularly pointed out in the appended claims.

It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate one embodiment of the invention, and together with the description, serve to explain the principles of the invention.

FIG. 1 is a partially exploded view of a syringe, according to an embodiment of the present invention.

FIG. 2 is a partially exploded view of a syringe, according to another embodiment of the present invention.

FIG. 3 is a schematic view of the device of FIG. 1 in a partially assembled configuration.

### DESCRIPTION OF THE EMBODIMENTS

Reference will now be made in detail to embodiments of the invention, an example of which is illustrated in the accompanying drawings. Wherever possible, the same reference numbers will be used throughout the drawings to refer to the same or like parts.

The present invention provides apparatus and processes for freezing and thawing cells in large volumes suitable for rapid expansion upon thawing. A large volume of cells frozen and thawed using a method according to the present invention have a survival rate of at least between 60%-90%, which is significantly higher than conventional cryopreservation methods for large cell masses.

The present invention also permits cryopreservation of the large volume of cells at higher densities (e.g., approximately 3.0×10⁷ cells/ml to 5.0×10⁸ cells/ml) without an animal serum. Freezing at such densities is accomplished through the addition of permeating cryoprotectants to the freeze media in greater than normal or high concentrations.

In addition, the present invention permits substantially direct inoculation of a bioreactor with the frozen cells. Specifically, the need for cell culture expansion after freezing has been eliminated. This saves time and reduces the potential for contamination.

According to one aspect of the present invention, a method and an apparatus for freezing a large cell mass at a high density is provided. As embodied herein, cells are separated from their previous media and densely packed by; for example, centrifugation at a relatively high force (i.e., a process known in the art as a “hard spin”). In preparation for freezing, the packed cells are then re-suspended in a suitable volume of biologically compatible freeze media.

Selection and preparation of the composition of the freeze media used to suspend and protect the cells during the freezing process involves consideration of several factors. The freeze media may comprise one or more additives including, but not limited to, animal serum (e.g. fetal calf serum (FCS) or fetal bovine serum (FBS)) and cryoprotectants (i.e., agents with high water solubility and low toxicity). Cryoprotectants introduced to the freeze media may enhance the survival of cells by limiting or preventing cell damage during the freezing and the thawing processes.

Cryoprotective agents, chemicals that reduce injury during freezing, are usually separated into two broad classes based on their ability to diffuse across cell membranes. Permeating cryoprotectants are able to move across cell membranes whereas non-permeating agents cannot. Permeating agents usually possess low molecular weight and high cell membrane permeability, and are believed to work by facilitating dehydration of the cell at early stages of cooling. As cooling proceeds, the permeating agents continue to diffuse into the cell, thereby depressing the intracellular freezing point by a colligative effect. Diffusion into the cell and replacement of the intracellular water protects against high osmotic pressure and prevents the cell's cytoskeleton from collapsing. Additionally, the permeating agent forms a shell that protects cell proteins from denaturation by vitrifying with any remaining water on the surface of the proteins.

Non-permeating cryoprotectants act by dehydrating the cell at high sub-freezing temperatures, thereby allowing them to be rapidly cooled, avoiding the injurious effects of slow cooling. These compounds are generally polymers that form extensive hydrogen bonds with water, reducing the water activity.

During the process of freezing, solute is rejected from the solid phase of the cell suspension solution, and an abrupt change in the concentration of the liquid portion of the solution is produced. In other words, freezing of the cell suspension (i.e., the cells suspended in freeze media) leads to the formation of ice, which causes a dramatic change in the concentration of water on one side of the cell membrane relative to the other. This dramatic change in concentration may create an osmotic pressure differential. A biological cell may respond to this transmembrane pressure differential by dehydrating itself to reach a new equilibrium state between the intracellular and extracellular solutions. At lower cooling rates, cells may be exposed to high sub-zero temperatures for long periods of time, causing the cells to become progressively dehydrated, which in turn may result in cell injury. In other words, if too much liquid were to leave a cell, the cell may shrivel and die.

Additionally, maintaining equilibrium at higher cooling rates may be difficult because the temperature is being lowered at a rate much greater than the rate at which water can diffuse out of the cell. Thus, as the temperature continues to drop, the liquid unable to diffuse out of the cell may begin to freeze intracellularly. Intracellular formation of ice is capable of causing substantial mechanical injury to a cell.

Therefore, a permeating cryoprotectant may be used to limit the incidence of cell damage and enhance the survival of cells during cryopreservation. DMSO may be preferred because of its high permeability to cell membranes. DMSO is capable of entering and exiting cells easily during freezing and thawing, and therefore reduces the incidence freeze damage.

The addition of a cryoprotectant in concentrations higher than those accepted as normal not only limits the incidence of cell damage and enhances the survival of cells, but also permits preservation of large cell masses in relatively small volumes (i.e., high densities) without the use of animal serums such as FCS. Currently, cells are usually frozen at densities between 1.0×10⁷ cells/ml and 5.0×10⁷ cells/ml in solutions containing, for example, 20% FCS and 10% DMSO. See Nobutaka Ninomiya et al., Large-Scale, High Density Freezing of Hybridomas and Its Application to High-Density Culture, 38 BIOTECHNOLOGY AND BIOENGINEERING 1110, 1110 (1991). However, because of growing concerns that substances such as FCS may present an unwanted source of contamination, it may be desirable to freeze large cell masses at higher densities without the use of substances such as FCS.

The present invention provides a method of preserving a high cell mass (e.g., a total cell number of between approximately 3.0×10⁸ cells and approximately 5.0×10⁹ cells in approximately 10 milliliters) at higher cell densities (e.g., freezing large cell masses at a density at least 10 times higher than that achieved by current methods) with a survival rate of at least 60%, and preferably about 90%. The method may be used with or without substances such as FCS. In order to achieve freezing of large cell masses at higher densities such as, for example, between 3.0×10⁷ cells/ml and 5.0×10⁸ cells/ml, without the use of substances such as FCS, the cells are frozen in a freeze media that contains a concentration of cryoprotectant higher than concentrations of cryoprotectant used in conventional methods. For example, conventional methods are only capable of freezing cells at higher densities (e.g., 1.5×10⁸ cells/ml) when the freeze media is supplemented with 20% FCS. See Nobutaka Ninomiya et al., Large-Scale, High Density Freezing of Hybridomas and Its Application to High-Density Culture, 38 BIOTECHNOLOGY AND BIOENGINEERING 1110, 1110 (1991). Additionally, methods that avoid the use of an animal serum have only been successful in freezing cells at a density of 5.0×10⁷ cells/ml in 10% DMSO. In contrast, the present method uses, for example, a DMSO concentration of between 15% to 25%, and preferably 20%.

The cryoprotectant concentration used in the present invention (e.g., 20% DMSO) permits preservation of cells at higher cell densities because the concentration of cryoprotectant creates an increase in the osmotic pressure differential between the intracellular and extracellular solutions. This pressure differential serves to dehydrate the cells by removing approximately 70% to 90% of the cells' water content. The increased concentration of the cryoprotectant also depresses the cells' freezing point and facilitates adequate cell dehydration. In addition, the increased concentration of cryoprotectant aids in protection of the intracellular proteins against denaturation. Thus, the incidence of intracellular freezing and ice formation is reduced and fewer cells are damaged as a result of intracellular ice.

Although DMSO in concentrations greater than those accepted as normal may present risks of toxicity to biological materials, embodiments of the present invention compensate for these potential risks by cooling and thawing cells at rates greater than those accepted as normal.

Furthermore, by increasing the concentration of the cryoprotectant from that used in conventional methods, the method according to the present invention may also compensate for the removal of any animal serum from the freeze media. The loss of the animal serum may be further compensated for, in some instances, by the addition of a small amount of a non-permeating cryoprotectant to the freeze media.

In some embodiments, such as processes involving one-step rapid freezing, it may be desirable to further include a small concentration (e.g., 1%-5%) of a non-permeating cryoprotectant. Non-permeating cryoprotectants aid in the dehydration of cells at higher temperatures, and are sometimes used to protect the cells' membranes. Examples of non-permeating cryoprotectants include, but are not limited to, sugars, dextran, ethylene glycol, polyvinyl pyrolidone, and hydroxyethyl starch.

In some instances, the cryoprotectant may be toxic to cells at normal temperatures. For example, DMSO toxicity is a function of temperature, the higher the temperature (e.g., greater than 4° C.) the more toxic it becomes. Therefore, it may be preferred to add pre-chilled freeze media containing DMSO rapidly to the cells just before the cells are frozen, i.e., when the temperature of the cells has been lowered to approximately 4° C.

Once the cells targeted for cryopreservation have been re-suspended in the freeze media, the entire solution may be transferred by any known process to an apparatus suitable for cryopreservation. For example, the solution may be transferred under a laboratory hood to a container made of a suitable biocompatible material having high purity and physical properties suitable for rapid freezing and long term cryostorage, such as, for example, cyclo-olefin-polymers or cyclo-olefin-copolymers.

Since cyclo-olefin-polymers and cyclo-olefin-copolymers possess a low permeability to gas and water vapor, they minimize adverse interactions with the cells. These materials do not have a glass transition point, and may be preferred because they are prevented from becoming brittle or fragile at low temperatures. Another exemplary advantage of using materials such as cyclo-olefin-polymers and cyclo-olefin-copolymers is that they possess a low coefficient of conduction, and are adaptable for use with various freezing processes, such as one-step freezing, rapid freezing, or direct freezing to the vapor phase.

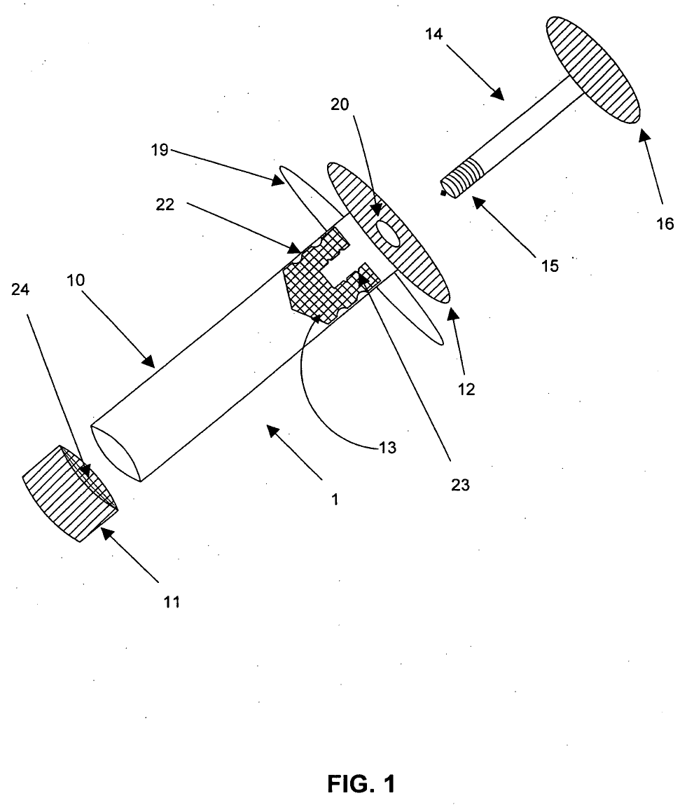

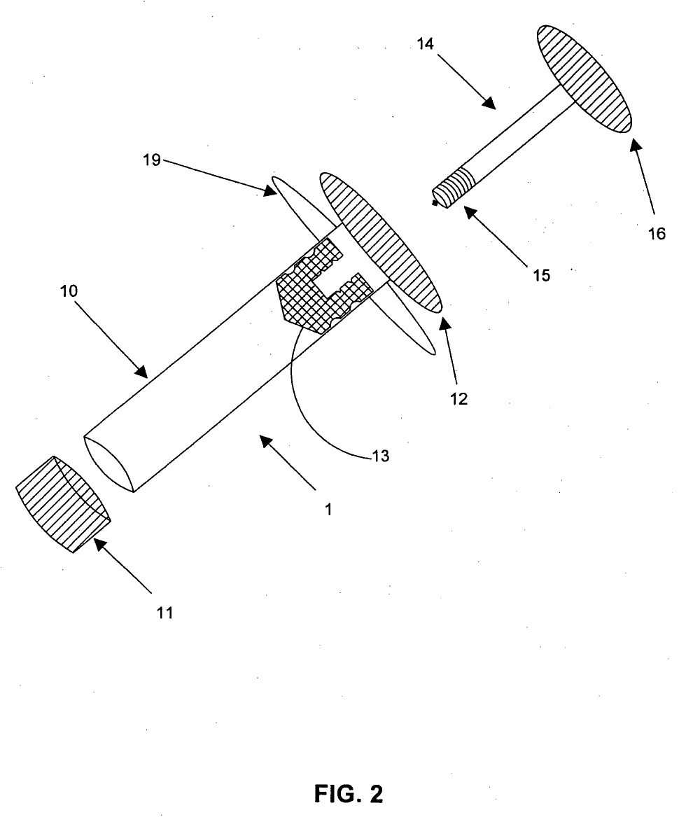

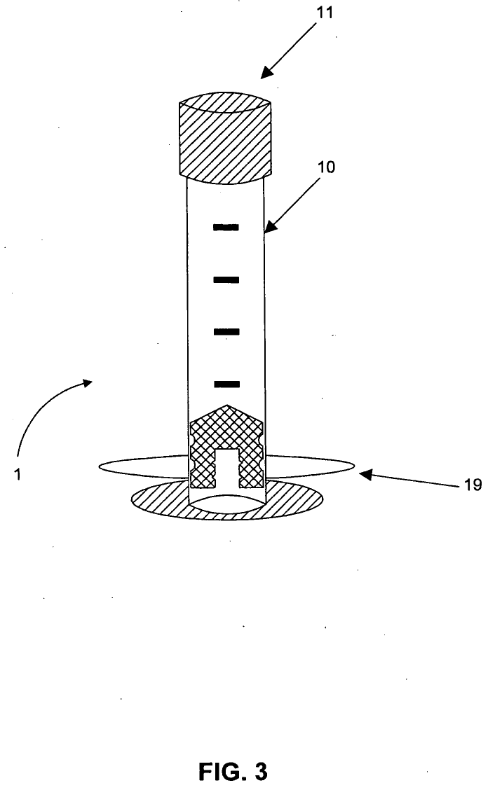

In one embodiment, the container in which the cells are stored during cryopreservation may be a syringe. FIGS. 1-3 depict certain configurations of an exemplary embodiment of such a syringe 1. As embodied herein and shown in FIGS. 2 and 3, syringe 1 includes a hollow, cylindrical body 10 having first and second open ends, and a finger flange 19 at one of the open ends. Syringe 1 also includes a first cap 11, a second cap 12, and a plunger 13 to be located inside the body 10 and adjacent to an end of the body. As shown in FIGS. 1 and 2, second cap 12 may or may not include an aperture 20 for facilitating connection between plunger 13 and plunger rod 14. Plunger 13 is configured to be used with a plunger rod 14 having a first end 15 and a second end 16. The plunger 13 may be made from an elastomer, such as halo-butyl synthetic rubber. The elastomeric portion may be prevented from direct contact with the contents of the syringe by a protective film, for example, an Ethylene Tetrafluoroethylene (ETFE) film, which covers the elastomeric portion. The film also may facilitate movement of the plunger 13 within body 10 of the syringe (i.e., overcoming friction). In embodiments where isolation of the syringe's contents is desired, the plunger 13 also may include ribs 22 to improve sealing between the plunger 13 and the syringe 1. The ribs 22 may have an approximate outer diameter of 15.3 mm to 15.4 mm.

The first end 15 of the plunger rod 14 may be configured to attach to plunger 13 by any known means. The second end 16 of the plunger rod 14 may be configured to facilitate longitudinal movement of the plunger rod 14. For example, the first end 15 of the plunger rod 14 may include screw threads adapted to be received in complimentary screw threads 23 provided in the plunger 13. The second end 16 of the plunger rod 14 may also include a flat circular surface or any other shape of any size suitable for actuating the plunger rod.

The syringe 1 and its components may be fabricated from any known biocompatible material suitable for freezing and long term cryostorage, and may have any desired cross-sectional shape and/or configuration. For example, the syringe 1 may have a substantially circular cross-section. The syringe 1 may also have one or more cross-sectional shapes, and/or configurations along its length, and any desired dimensions suitable to cryopreservation and/or any subsequent processes. In one embodiment, the syringe 1 may have dimensions adapted for inoculation of a bioreactor or similar device, for example, an overall length of approximately 84 mm, a body having an outside diameter of 19 mm, and a wall thickness of approximately 1.5 mm. It should be understood that the syringe 1 may be used for any process requiring the storage, and/or the transfer of cells from one source to another. In addition, the syringe 1 may be used with any type of cells desired, and may be used in an environment that is relatively fluid filled, or that is relatively dry.

By way of example, the syringe's first cap 11 may be attached to: the body 10 by any suitable means known, for example, with threads 24 or by snap fitting elements. Sealing between cap 11 and body 10 may be provided by any suitable means known, including but not limited to, o-rings, gaskets, and plug and chamfer seals. Next, the body 10 may be filled with a solution containing cells targeted for cryopreservation. Subsequently, the second cap 12 and a plunger 13 may then be attached to the body 10 by any suitable means known.

Alternatively, plunger 13 may already be positioned within the body 10, and cap 11 or cap 12 attached to body 10. The syringe can then be filled with the solution containing the cells targeted for cryopreservation prior to the attachment of the remaining cap 11 or cap 12. Any other method that allows for substantially sterile filling of body 10 may also be used.

Once the solution containing the cells targeted for cryopreservation has been placed in a storage receptacle (e.g., a syringe or other vial) suitable for cryopreservation, the freezing process may begin. For example, one or more receptacles containing cells in freezing media may be placed in a suitable storage container for cooling, such as a Styrofoam box or a controlled-rate freezer. The receptacles may then be cooled to an appropriate temperature suitable for cryopreservation, and/or cryostorage. For example, the receptacles may first be cooled at a controlled rate of freezing, such as at a rate of approximately 1° C./minute, to a temperature of approximately −80° C. Subsequently, the samples may then be transferred to vapor phase liquid nitrogen for storage, where they are further cooled to a temperature of approximately −130° C. or below.

In other instances, it may be desirable to alter the exemplary method disclosed by combining and/or eliminating one or more steps of the method. For example, based on the dimensions (i.e., wall thickness) and/or conduction rate of the container chosen, the step of first cooling the container to a temperature of approximately −80° C. may be eliminated. In such an instance, the container must possess a low thermal conductivity and may comprise an overall length of approximately 84 mm, a body having an outside diameter of 19 mm, and a wall thickness of approximately 1.5 mm. Instead of being subjected to a two-step freezing process, the container, along with the samples contained therein, may be directly cooled to a temperature of approximately −130° C. or below. In such a method, the cooling rate is faster than the two-step process, for example, approximately 8° C./minute.

It is understood that the cooling of the samples may be achieved by any means suitable and/or known in the art. For example, the cells may be placed in a freezer or placed in a tank containing a-cooling fluid (e.g., nitrogen vapor).

According to another aspect of the present invention, a method of rapidly thawing the cryopreserved cells will be described.

When the cryopreserved cells are required for use, the frozen samples may be thawed by any means suitable and/or known in the art. For example, the samples may be removed from the freezer or liquid nitrogen and the receptacles may be placed on dry ice to begin the thawing process.

In an embodiment in which the cells were stored in a syringe as shown in FIGS. 1-3, the outside of the syringe may be sprayed with alcohol, or any other substance suitable to disinfect and/or sterilize the outer surface of the storage receptacle. Next, the frozen contents of the syringe are emptied directly into a thawing receptacle such as, for example, a spinner culture containing, for example, growth media at 37° C. by, for example, removing cap 11, connecting the plunger rod 14 to the plunger 13 through aperture 20 provided in cap 12, and actuating the plunger to eject the frozen cells from the syringe and into the spinner. Alternatively, to reduce the potential of contamination, the cap 12 may not be provided with an aperture 20 (see FIG. 2). In such instances, prior to connecting the plunger rod 14 to plunger 12, the cap 12 must first be removed.

Once in the spinner, the growth media rapidly dilutes the DMSO and negates its toxicity as the DMSO and cells thaw. Subsequently, the cells are separated from the freeze media, and are then ready for further processing, such as being used for inoculation of a bioreactor. Ten milliliters of frozen cells and media thaw in approximately 43 seconds, which is significantly faster than the thawing time for other conventional methods. Although this method of rapid thawing has been described in conjunction with the use of a syringe container for cell storage, any other suitable container in which cells can be cryopreserved and subsequently removed from the container in their frozen state may be used.

One exemplary advantage of using a method according to the present invention to thaw cryopreserved cells is reduction in the incidence of recrystallization in the intracellular and/or extracellular solutions during thawing. The present invention's method of thawing prevents the formation and/or growth of potentially damaging ice crystals by utilizing a rapid rate of warming. The rapid rate of warming, as compared to current methods, makes it difficult for small ice crystals that may have been formed during the freezing process to grow into harmful large ice crystals (i.e., recrystallization) by reducing the time needed to go through the critical zone of approximately −60° C. to approximately −15° C.

The cryopreservative (e.g., DMSO) will normally be used in a solution at a concentration sufficient to assure acceptable survival without being toxic in subsequent use, for example, when transfused. The amount of cryopreservative used may also be dependent on the type of cells being preserved. Moreover, treatment conditions, such as pre- or post-storage dilution with suitable buffers or cell culture media, may be desirable.

In the preceding detailed description, reference has been made to the accompanying drawings that form a part hereof, and in which are shown by way of illustration specific embodiments, in which the invention may be practiced. These embodiments have been described in sufficient detail to enable those skilled in the art to practice the invention and it is to be understood that other embodiments may be utilized and that logical, mechanical, and chemical changes may be made without departing from the spirit or scope of the invention. To avoid detail not necessary to enable those skilled in the art to practice the invention, the description omits certain information known to those skilled in the art.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. An apparatus for storing and dispensing cryopreserved cells, comprising:
a body having an open first end and an open second end;
a first cap configured to removably attach to the open first end;
a second cap configured to removably attach to the open second end;
a plunger portion contained within the body and adjacent to one of said open ends; and
a plunger rod configured to be connected to the plunger portion,
wherein at least a portion of the apparatus is made from a biocompatible material.

2-10. (canceled)

11. A method of rapidly freezing cells, comprising:
acquiring a desired quantity of cells for cryostorage;
suspending the acquired cells in chilled freeze media containing a permeating cryoprotectant, wherein the freeze media is at a temperature of approximately 0° C. to 4° C.;
placing the cells and freeze media in an apparatus configured to store and dispense cryopreserved cells, wherein at least a portion of the apparatus is made from a biocompatible material; and
rapidly cooling the apparatus containing the cells and chilled freeze media to a temperature of −130° C. or below at a rate of approximately 8° C./minute.

12-15. (canceled)

16. A method of rapidly thawing cryopreserved cells, comprising:
retrieving a storage apparatus containing frozen media and cells having an approximate temperature of −130° C. or below; and
transferring the frozen media and cells from the storage apparatus to a thawing vessel containing growth media at a temperature of approximately 37° C. to thaw the cells.

17-18. (canceled)

19. A method for cryostoring cells comprising:
acquiring a desired quantity of cells for cryostorage;
placing the acquired cells in chilled freeze media containing a permeating cryoprotectant;
storing the cells and freeze media in an apparatus suitable for cryostorage, wherein the apparatus is configured to store and dispense cryopreserved cells and comprises:
a body having an open first end and an open second end;
a first cap configured to removably attach to the open first end;
a second cap configured to removably attach to the open second end;
a plunger portion contained within the body and adjacent to one of said open ends; and
a plunger rod configured to be connected to the plunger portion, wherein at least a portion of the apparatus is made from a biocompatible material; and
cooling the apparatus to an approximate temperature of −130° C. or below.

20-27. (canceled)

28. A method for inoculating a bioreactor with cryopreserved cells comprising:
acquiring a desired quantity of cells for cryostorage;
placing the acquired cells in chilled freeze media containing a permeating cryoprotectant;
storing the cells and freeze media in an apparatus configured to store and dispense cryopreserved cells, wherein the apparatus comprises:
a body having an open first end and an open second end;
a first cap configured to removably attach to the open first end;
a second cap configured to removably attach to the open second end;
a plunger portion contained within the body and adjacent to one of said open ends; and
a plunger rod configured to be connected to the plunger portion, wherein at least a portion of the apparatus is made from a biocompatible material;
cooling the apparatus to an approximate temperature of −130° C. or below;
subsequent to cooling the apparatus to an approximate temperature of −130° C. or below, transferring the frozen media and cells from the apparatus to a thawing vessel containing growth media at a temperature substantially warmer than 0° C.; and
inoculating a bioreactor with the cells from the thawing vessel.

29-38. (canceled)

39. A composition for cryopreserving a large cell mass at a high density, comprising:
a freeze media including a permeating cryoprotectant, wherein the concentration of the permeating cryoprotectant is sufficient to permit the cells to be stored at a density greater than 1.5×10⁸ cells/ml; and
a large volume of cells, between 3.0×10⁸ cells and 5.0×10⁹ cells, to be stored.

40-51. (canceled)

52. A method of freezing a large cell mass at a high density, comprising:
suspending a large cell mass in a freeze media containing a permeating cryoprotectant, wherein the concentration of the permeating cryoprotectant is sufficient to permit the cells to be stored at a density greater than 1.5×10⁸ cells/ml;
placing the cells and freeze media in a storage apparatus; and
cooling the cells and freeze media to a temperature at or below approximately −130° C.

53-62. (canceled)

63. A method of rapidly thawing a large, frozen cell mass, comprising:
retrieving a storage apparatus containing a frozen cell mass and freeze media; and
transferring the frozen cell mass and freeze media from the storage apparatus to a thawing vessel containing growth media at a temperature of approximately 37° C. to thaw the cells.

64-65. (canceled)

66. A composition for cryopreserving a large cell mass at a high density, comprising:
a freeze media including 20% DMSO, wherein the freeze media does not include animal serum, and wherein the concentration of the DMSO is sufficient to permit the cells to be stored at a density greater than 3.0×10⁷ cells/ml; and
a large volume of cells to be stored.

67. A method of freezing a large cell mass at a high density, comprising:
suspending a large cell mass in a freeze media containing 20% DMSO, wherein the freeze media does not include animal serum and wherein the concentration of the DMSO is sufficient to permit the cells to be stored at a density greater than 3.0×10⁷ cells/ml;
placing the cells and freeze media in a storage apparatus; and
cooling the cells and freeze media to a temperature at or below approximately −130° C.
